(19) United States
(12) Patent Application Publication
(10) Pub. No.: US 2025/0136633 A1
(43) Pub. Date: May 1, 2025

(54) TELOMERE-TARGETING PHOSPHATIDYL-THIO CONJUGATES

(71) Applicant: MAIA BIOTECHNOLOGY, INC., Chicago, IL (US)

(72) Inventor: Sergei Gryaznov, San Mateo, CA (US)

(73) Assignee: MAIA BIOTECHNOLOGY, INC., Chicago, IL (US)

(21) Appl. No.: 18/930,974

(22) Filed: Oct. 29, 2024

Related U.S. Application Data
(60) Provisional application No. 63/546,242, filed on Oct. 29, 2023.

(51) Int. Cl. C07H 19/20; A61K 31/7076; A61P 35/00

(52) U.S. Cl. CPC C07H 19/20; A61K 31/7076; A61P 35/00

(57) ABSTRACT

Disclosed herein are telomere-targeting phosphatidyl-thio conjugates that target telomers in cancer cells and methods for using the telomere-targeting phosphatidyl-thio conjugates compounds to treat cancers.

### CROSS-REFERENCE TO RELATED APPLICATIONS

This application claims priority under 35 U.S.C § 119, based on U.S. Provisional Application Ser. No. 63/546,242, filed on 29 Oct. 2023, and entitled, “Telomere-Targeting Phosphatidyl-Thio Conjugates”

### FIELD OF THE INVENTION

The present disclosure relates to the fields of medicine, pharmacology, molecular biology and oncology. More particularly, the disclosure relates to telomere-targeting phosphatidyl-thio conjugates with anticancer activity.

### BACKGROUND OF THE DISCLOSURE

Immunotherapies have revolutionized the treatment of many cancers in the immuno-oncology field (Brahmer et al., 2012; Hodi et al., 2010; Ribas and Wolchok, 2018; Topalian et al., 2012). The most commonly used immunotherapies are PD-L1/PD-1 checkpoint blockades that have been approved by the FDA for advanced cancers such as melanoma, non-small cell lung cancer, breast cancer, cervical cancer, colon cancer, head and neck cancer, Hodgkin lymphoma, liver, cancer, lung cancer, renal cell cancer, stomach cancer, rectal cancer, and any solid tumor that is not able to repair errors in its DNA that occur during replication (Garon et al., 2015; Ribas et al., 2016; Rizvi et al., 2015b; Socinski et al., 2018; National Cancer Institute). Despite the success of immunotherapies, many patients do not respond well to these therapies due to the immune suppressive tumor microenvironment, tumor immunogenicity and the emergence of primary and adaptive resistance (Chen and Han, 2015; Gide et al., 2018). Although recent studies show that the abundance of tumor mutations and neoantigens partially dictate cancer patient responses to checkpoint blockade, there are still considerable numbers of patients with high mutations and neoantigens that do not respond well (Le et al., 2017; Mandal et al., 2019; Rizvi et al., 2015a), suggesting neoantigens are not sufficient for provoking anti-tumor immune responses. Therefore, there is an urgent need to identify other factors for better immune responses and to develop new approaches to improve patient overall survival.

The generation of effective anti-tumor adaptive immune responses require tumor antigen presentation by antigen presenting cells, whose activation heavily rely on adequate innate sensing. Innate sensing is often provided by danger signals such as high mobility group box 1 protein, extracellular ATP and tumor DNAs released from stressed tumor cells (Kroemer et al., 2013; Pitt et al., 2017). Recent studies highlight the importance of cytosolic DNA sensing in radiation and DNA damaging therapies (Deng et al., 2014; Sen et al., 2019). The presence of DNA in the cytoplasm, for example, in the form of micronuclei (small DNA containing organelles) that lose nuclear envelop membranes can trigger immune responses. Micronuclei are the products of chromosome damage as a result of genotoxic stress and chromosome mis-segregation during cell division (Fenech et al., 2011). The cytosolic DNA sensor cGAS recognizes micronuclei and converts GTP (guanosine triphosphate) and ATP (adenosine triphosphate) into second messenger cGAMP (cyclic GMP-AMP) (Wu et al., 2013). Then the adaptor protein Stimulator of IFN Gene (STING) binds to cGAMP (Ablasser et al., 2013; Diner et al., 2013; Gao et al., 2013; Zhang et al., 2013). This complex process activates TANK-binding kinase 1 (TBK1) and IFN regulatory factor 3 (IRF3) (Liu et al., 2015; Tanaka and Chen, 2012) and further activates the downstream transcription of type I IFNs and other cytokines (reviewed in (Li and Chen, 2018)), which ultimately increases innate sensing.

Eukaryotic linear chromosomes are capped by special structures called telomeres (TTAGGG), which are essential to maintain chromosomal stability (reviewed in (Blackburn, 1991)). Telomeres constitute the final ˜10 kb of all human chromosomes and the final 12-80 kb of all mouse chromosomes (Lansdorp et al., 1996; Zijlmans et al., 1997). In all somatic human cells, telomeres shorten with each cell division due to the end replication problem and the absence of a telomere maintenance mechanism (reviewed in Greider, 1996). However, unicellular eukaryotes, germline cells and immortal cancer cells maintain their telomeres at a constant length almost always by activating the enzyme telomerase (Greider and Blackburn, 1985; McEachern and Blackburn, 1996; Morin, 1989; Nakamura et al., 1997; Singer and Gottschling, 1994; Yu et al., 1990). Telomerase is a reverse transcriptase enzyme that elongates telomeres by adding TTAGGG repeats to the ends of chromosomes and is expressed in ˜90% of human tumors, but not in most normal cells (Shay and Bacchetti, 1997). Therefore, telomerase is an attractive target to develop anti-cancer therapies.

The nucleoside analogue, 6-thio-2′-deoxyguanosine (6-thio-dG), is a new and effective therapeutic approach in the cancer field. Its incorporation into de novo synthesized telomeres by telomerase is known to induce damage on telomeric DNA (Mender et al., 2015a). This results in rapid tumor shrinkage or growth arrest in many tumor-derived xenograft models with minimal side effects (Mender et al., 2018; Sengupta et al., 2018; Zhang et al., 2018). The most important advantage of this telomere-targeted therapy over direct telomerase inhibitors is that 6-thio-dG does not have a long lag period for tumor killing effects. Additionally, it does not directly inhibit telomerase but is preferentially recognized by telomerase over other polymerases and incorporated into the telomeres resulting in an immediate DNA chain termination. Importantly, its effect is independent of initial telomere length by hijacking tumor telomerase to make unstable telomeres (Mender et al., 2015b). Disclosed herein are THIO-Lipid conjugates that are useful for telomere-targeted therapy.

### SUMMARY OF THE DISCLOSURE

An aspect of the disclosure includes THIO-Lipid conjugated prodrug comprising a compound of Formula I

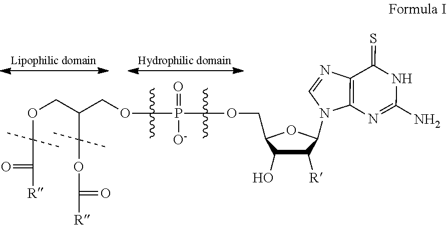

wherein R′ is H or OH and R″ is C3-C17.

In some embodiments, the compound of Formula I is selected from one of more of the following compounds:
5′-(1,2-dimyristoyl-sn-glycero-3-phospho)-6-thio-2′-deoxyguanosine,
5′-(1,2-dilauroyl-sn-glycero-3-phospho)-6-thio-2′-deoxyguanosine,
5′-(1,2-dilauroyl-sn-glycero-3-phospho)-6-thio-guanosine,
5′-(1,2-dioctanoyl-sn-glycero-3-phospho)-6-thio-2′-deoxyguanosine,
5′-(1,2-dioctanoyl-sn-glycero-3-phospho)-6-thio-guanosine,
5′-(1,2-dihexanoyl-sn-glycero-3-phospho)-6-thio-2′-deoxyguanosine,
5′-(1,2-dibutyryl-sn-glycero-3-phospho)-6-thio-2′-deoxyguanosine,
5′-(1,2-dihexanoyl-sn-glycero-3-phospho)-6-thio-guanosine,
5′-(1,2-dipalmitoyl-sn-glycero-3-phospho)-6-thio-2′-deoxyguanosine,
5′-(1,2-distearoyl-sn-glycero-3-phospho)-6-thio-2′-deoxyguanosine, and
5′-(1,2-dioleoyl-sn-glycero-3-phospho)-6-thio-2′-deoxyguanosine.

In some embodiments, the THIO-Lipid conjugate has the following structure:

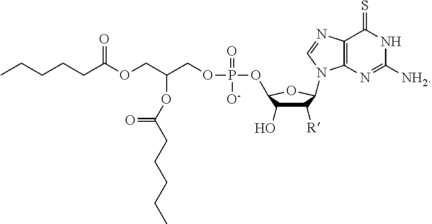

In some embodiments the THIO-Lipid conjugated prodrug is a pharmaceutical composition.

In some embodiments the pharmaceutical composition is a formulation that includes at least one pharmaceutically acceptable excipient.

In some embodiments, the Thio Lipid conjugated prodrug is used in a method of treating a subject who has cancer, comprising administrating to the subject a compound of any one of claims 1-5, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

### BRIEF DESCRIPTION OF THE DRAWINGS

FIG. 1 shows the dose response curves for L6-Phosphatidyl-THIO Conjugate in Cancer Cells: A) HT-29 cell viability after treatment with Compound L6; B) CT-26 Cell viability after treatment with Compound L6; C) HeLa Cell viability after treatment with Compound L6 and D) EC₅₀ values (μM) of 6-thio-dG and Compound L6 in various cell lines.

FIG. 2 shows TIF Formation Induced by Phosphatidyl Conjugates in Hela Cells—A) Hela cells treated with 1 μM Phosphatidyl Conjugates; B) HeLa cells treated at 3 μM Phosphatidyl Conjugates.

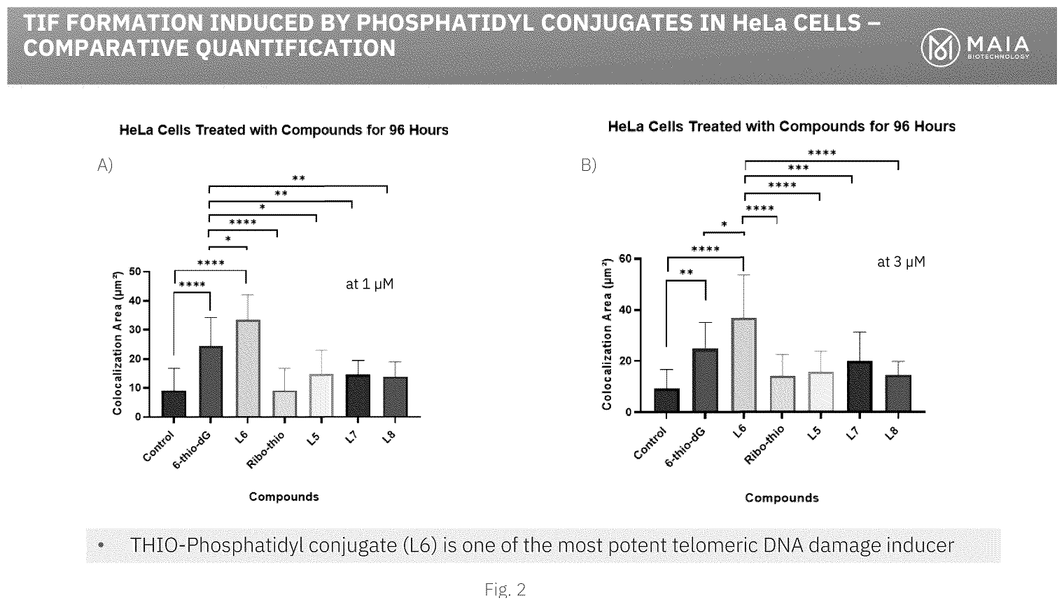

FIG. 3—Shows in vivo anticancer activity of phosphatidyl-THIO conjugate (L6) in HT 29 cells-derived xerograph colorectal carcinoma model at two different concentrations.

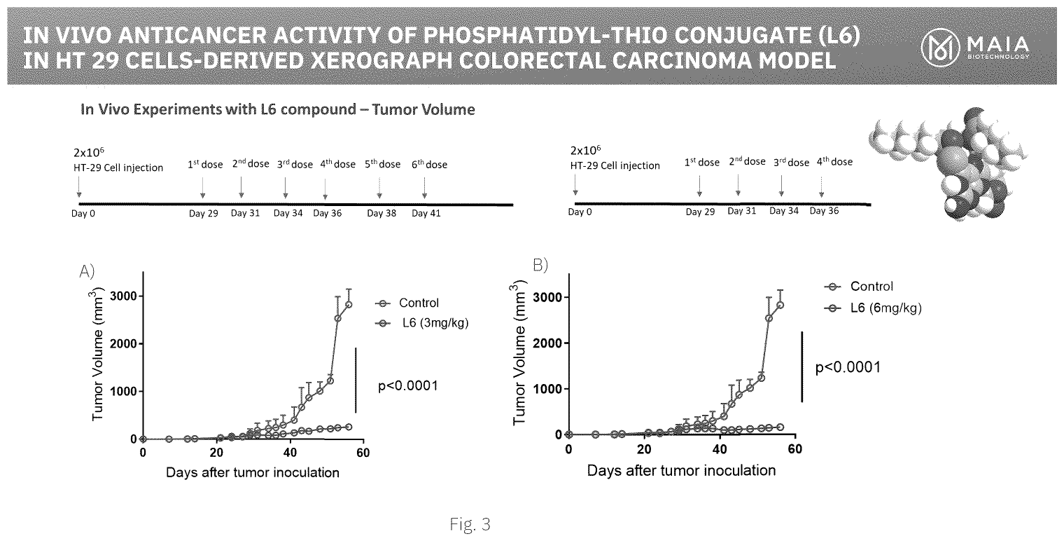

FIG. 4—shows Phosphatidyl-THIO Conjugate L6 Activity in Immune Competent Model—CT26 murine colon carcinoma model—with L6 alone and L6 with an anti-PDL1.

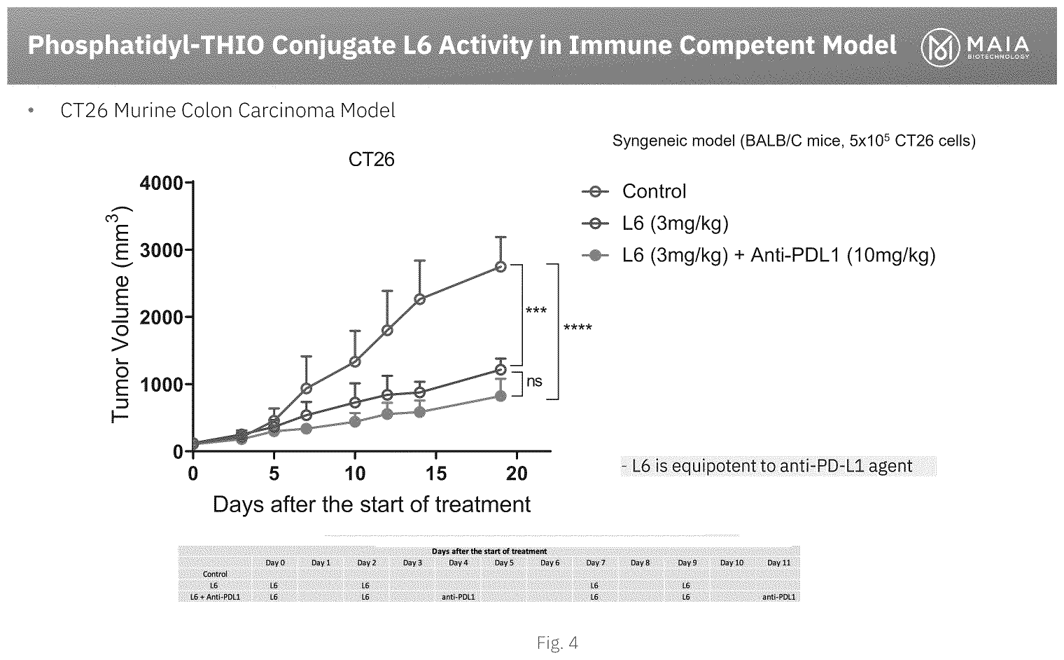

### DETAILED DESCRIPTION OF THE EMBODIMENTS

An aspect of the present disclosure are prodrugs of 6-thio-2′-deoxyguanosine (6-thio-dG or THIO) and 6-thio-2′ guanosine (ribo-THIO).

In some embodiments, the prodrugs contain two domains—a lipophilic domain and hydrophilic domain as illustrated in Formula I:

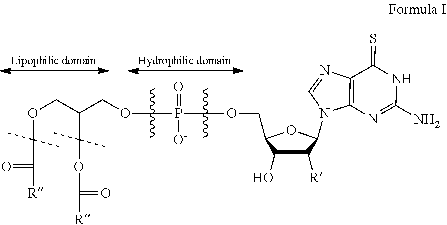

wherein R′ is H or, OH and R″ is C₃-C₁₇.

The anticancer activity of these prodrugs may be defined by an optimal ratio between the conjugate's two domains. The lipid group is involved in delivery and PK whereas the nucleoside pharmacophore is involved in anticancer, and antiviral activity.

Also described in the Example Section, several 5′-phosphatidyl derivatives of THIO and ribo-THIO were prepared for the first time. In addition, a one-pot enzymatic method for liponucleotides synthesis is described. The obtained conjugates were investigated for their anticancer activity in vitro and in vivo.

The following new phosphatidyl-thio conjugates were prepared and evaluated (Table 1):

TABLE 1
Phosphatidyl-THIO conjugates

| | | |
|---|---|---|
| 1 | 5′-(1,2-dimyristoyl-sn-glycero-3-phospho)-6- | C14-6SdG |
| | thio-2′-deoxyguanosine | |
| 2 | 5′-(1,2-dilauroyl-sn-glycero-3-phospho)-6- | C12-6SdG |
| | thio-2′-deoxyguanosine | |
| 3 | 5′-(1,2-dilauroyl-sn-glycero-3-phospho)-6- | C12-6SriboG |
| | thio-guanosine | |
| 4 | 5′-(1,2-dioctanoyl-sn-glycero-3-phospho)-6- | C8-6SdG |
| | thio-2′-deoxyguanosine | |
| 5 | 5′-(1,2-dioctanoyl-sn-glycero-3-phospho)-6- | C8-6SriboG |
| | thio-guanosine | |
| 6 | 5′-(1,2-dihexanoyl-sn-glycero-3-phospho)-6- | C6-6SdG |
| | thio-2′-deoxyguanosine | |
| 7 | 5′-(1,2-dibutyryl-sn-glycero-3-phospho)-6-thio- | C4-6SdG |
| | 2′-deoxyguanosine | |
| 8 | 5′-(1,2-dihexanoyl-sn-glycero-3-phospho)-6- | C6-6SriboG |
| | thio-guanosine | |
| 9 | 5′-(1,2-dipalmitoyl-sn-glycero-3-phospho)-6- | C16-6SdG |
| | thio-2′-deoxyguanosine | |
| 10 | 5′-(1,2-distearoyl-sn-glycero-3-phospho)-6-thio- | C18-6SdG |
| | 2′-deoxyguanosine | |
| 11 | 5′-(1,2-dioleoyl-sn-glycero-3-phospho)-6-thio- | C18:01-6SdG |
| | 2′-deoxyguanosine | |

C6 Phosphatidyl conjugates for both 2′-deoxy- and 2′-hydroxy-6-thioguanosines show the highest anticancer activity, which is similar, or superior to that for the free pharmacophores.

Saturated highly lipophilic C18 derivative is the least active compound possibly because of cellular membrane conjugate retention.

Interestingly, mono-unsaturated C18:1 (oleoyl) conjugate demonstrates essentially recovered cytotoxic activity.

Treatment of cancer cell cells with C6-THIO conjugate results in efficient induction of telomere dysfunction-induced foci (TIF)-positive circulating tumor cells and micronuclei formation (Data not shown)

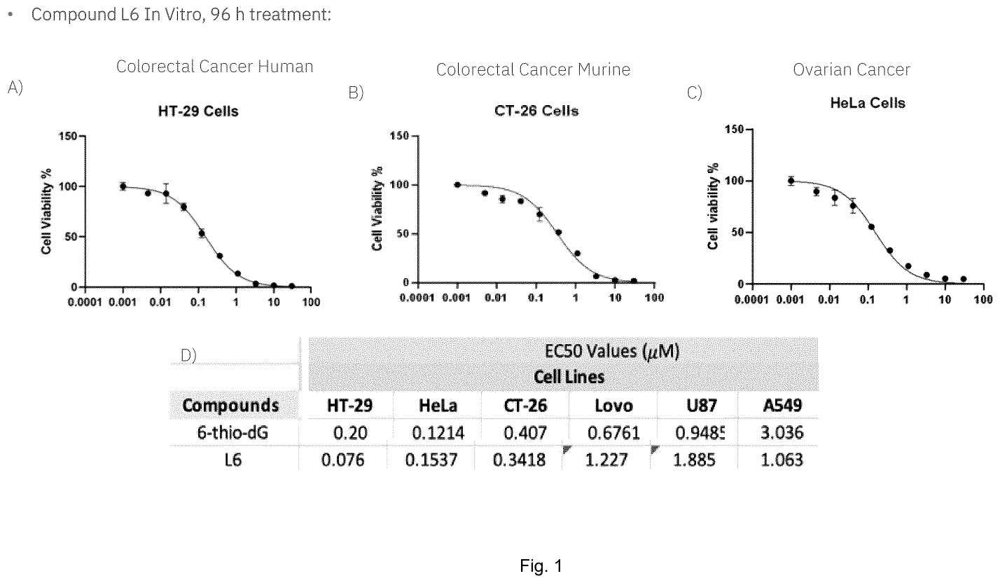

Shown FIG. 1 are the representative dose response curves of C6-Phosphatidyl-THIO conjugate in HT-29 cells, CT-26 Cells and HeLa Cells. The structure of C6-Phosphatidyl-THIO is shown below:

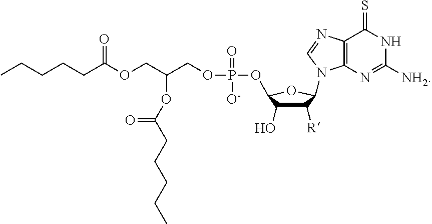

The in vitro activity of Phosphatidyl-THIO conjugates were tested in HT29 Colorectal Carcinoma Cells (CRC) (Table 2)

TABLE 2
In vitro anticancer activity of phosphatidyl-THIO conjugates: HT 29 CRC cells

| | | EC₅₀ Values, (μM) | | | | | | | | |
|---|---|---|---|---|---|---|---|---|---|---|
| Pharmacophore* | | Phosphatidyl Lipid | | | | | | | | |
| — | C₃ | C₄ | C₆ | C₈ | C₁₀ | C₁₂ | C₁₄ | C₁₆ | C₁₈ | C_{18:1} |

| | | | | | | | | |
|---|---|---|---|---|---|---|---|---|
| 6-thio-dG | 0.26 | 0.08 | 0.35 | 0.48 | 0.49 | n.d** | 12.35 | 0.46 |
| Ribo-THIO | | 0.20 | 0.26 | 0.60 | | | | |

*Where the EC₅₀ values for unconjugated pharmacophores were 0.20 μM and 0.14 μM, for 6-thio-dG and riboTHIO, respectively
**Not determined due to the compound's solubility

Another aspect of the disclosure comprises a method for treating a cancer including a resistant, refractory and/or metastatic cancer, the method comprising administering to the subject a first amount or dose of one or more phosphatidyl-THIO conjugate compounds disclosed herein in a therapeutically effective amount that is effective to shorten telomere length; reduce size of a tumor; reduce growth rate of a tumor; reduce incidence of metastasis; eliminating metastasis, promote an immune response; reduce progression of the cancer; increase lifespan of the subject; or a combination thereof.

In embodiments of this aspect, the one or more phosphatidyl-THIO conjugate compounds disclosed herein are given to a subject in need thereof in combination with one or more other cancer therapies wherein the combination is effective to shorten telomere length; reduce size of a tumor; reduce growth rate of a tumor; reduce incidence of metastasis; promote an immune response; reduce progression of the cancer; increase lifespan of the subject; or a combination thereof.

The term “subject” as used herein refers to either a human or non-human, such as primates, mammals, and vertebrates. According to some embodiments, the subject is a human.

The term “subject in need of such treatment or in need thereof” as used herein refers to (i) a patient who suffers from a cancer; and (ii) a subject who will be administered a one or more phosphatidyl-THIO conjugate compounds disclosed herein.

The term “therapeutically effective dose” as used herein refers to a dose (i.e., dose and frequency of administration) that eliminates, reduces, or prevents the progression of a particular disease manifestation in a percentage of a population. An example of a commonly used therapeutic component is the ED₅₀, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The terms “therapeutically effective amount”, an “amount effective”, or “pharmaceutically effective amount” of an active agent are used interchangeably to refer to an amount that is sufficient to provide the intended benefit of treatment. An effective amount of an active agent that can be employed according to the described invention generally ranges from generally about 0.01 mg/kg body weight to about 100 g/kg body weight. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods.

The term “treat” or “treating” includes abrogating, substantially inhibiting, slowing or reversing the progression of a disease, condition or disorder, substantially ameliorating clinical symptoms of a condition, substantially preventing the appearance of clinical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

According to some embodiments, the cancer treated using the one or more phosphatidyl-THIO conjugate compounds disclosed herein is, without limitation, a carcinoma, a sarcoma, a leukemia, a lymphoma/myeloma or a brain/spinal cord cancer. According to some embodiments, the cancer comprises a solid tumor comprising tumor cells, a metastatic cancer comprising metastatic tumor cells, or a combination thereof.

In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. A tumor can comprise a malignant or benign growth.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The cancer may be lung cancer, colon cancer, liver cancer, or brain cancer. The lung cancer may be non-small cell lung cancer. The colon cancer may be colorectal cancer. The liver cancer may be hepatocellular cancer.

Anticancer activity of the one or more phosphatidyl-THIO conjugate compounds disclosed herein, was evaluated in vitro in various cancer cell lines, according to commonly used procedures for assessment of compounds cytotoxicity, as out lined in the following publications: Mender, I., Gryaznov, S., Dikmen, Z. G., Wright, W. E., & Shay, J. W. (2015). Induction of telomere dysfunction mediated by the telomerase substrate precursor 6-thio-2′-deoxyguanosine. Cancer Discovery, 5, 82-95; Mender et al., (2020). Telomere Stress Potentiates STING-Dependent Anti-tumor Immunity. Cancer Cell, 38, 400-411.

### Pharmaceutical Formulations and Routes of Administration

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Aqueous compositions of the present disclosure comprise an effective amount of the drug dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the agents of the compositions.

The active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or intratumoral or regional to a tumor, such as in the tumor vasculature. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The actual dosage amount of a composition in accordance with certain embodiments of the present disclosure administered to subject can be determined by physical and physiological factors such as the specific compound employed, the age, general health of the subject, diet, body weight, severity of condition, the type of disease being treated, previous or concurrent therapeutic interventions, idiopathy of the patient, absorption rates, distribution rates, inactivation rates, excretion rates, time of administration, the route of administration, and on the, judgment of the person supervising the administration. Depending upon the dosage and the route of administration, the number of administrations of a preferred dosage, and/or an effective amount may vary according to the response of the subject. The active ingredient may be administered at once, or may be divided into a number of smaller doses to be administered at varying intervals of time. As such, it is understood that for any particular subject, specific dosage regimens could be adjusted over time according to the individual need and the professional judgment of the person administering or supervising the administration of the compositions, and that the concentration ranges set forth herein are exemplary only and are not intended to limit the scope or practice of the claimed composition.

The phosphatidyl-THIO conjugates disclosed herein are included in the pharmaceutically acceptable carrier or diluent in an amount sufficient to deliver to a patient a therapeutically effective amount for the desired indication, without causing serious toxic effects in the patient treated. A preferred dose of the phosphatidyl-THIO conjugates for all of the herein-mentioned conditions is in the range from about 10 ng/kg to 100 mg/kg, preferably 0.1 to 50 mg/kg per day, more generally 0.5 to about 25 mg per kilogram body weight of the subject per day. By way of non-limiting example, a typical dosage can range from 50.01-20% wt/wt in a suitable carrier. Similarly, the compound can be administered in any suitable unit dosage form, including, but not limited to, one containing less than 1 mg, 1 mg to 3000 mg, or 5 mg to 1000 mg of phosphatidyl-THIO conjugates disclosed herein.

Compositions may be administered on an ongoing or continuous basis; on an as needed basis; or 1, 2, 3, 4, 5, 6, 5, 8, 9, 10, 11, 12, 13, 14, 15, 16, 17, 18, 19, 20 or more times including but not limited to one containing less than 1 mg.

### Combined Therapy

In order to increase the effectiveness a treatment, it may be desirable to combine compositions of the present disclosure with a second treatment or pharmaceutical composition. For example, a method of use can further include administration of a second pharmaceutical composition comprising an anti-cancer agent or other agent effective in the treatment of hyperproliferative disease. An anti-cancer agent can negatively affect cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, a second pharmaceutical composition can be administered in an effective amount or combined effective amount to kill or inhibit proliferation of certain cells.

In some embodiments, a method of treatment can comprise a simultaneous co-administration. This process may involve administration at the same time or sequentially. Co-administration can be achieved by contacting the cell with a single composition or pharmaceutical formulation that includes one or more of the phosphatidyl-THIO conjugates and another anti-cancer agent, or by contacting the cell with two distinct compositions or formulations, at the same time, wherein one composition includes one or more of the phosphatidyl-THIO conjugates compounds disclosed herein and the other includes the second agent(s). Similarly, two compositions can be administered not at the same time but in temporal proximity to each other, e.g., on the same day or within the same week.

In other embodiments, a method of treatment can comprise a first stage wherein a pharmaceutical composition comprising one or more of the phosphatidyl-THIO conjugates is administered and a second stage where a second pharmaceutical composition is administered. The first stage and the second stage may be sequential in time, spaced apart in time (minutes, days, weeks, or months), or overlapping in time. In addition, the sequential order of treatment stages can be reversed or repeated.

To be sure, any combination of treatment stages may be employed. By way of example, administration one or more of the phosphatidyl-THIO conjugate compounds disclosed herein is “A” and the treatment with a secondary agent is “B”:

A/B/A B/A/B B/B/A A/A/B A/B/B B/A/A A/B/B/B B/A/B/B

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

In the context of the present disclosure, it is contemplated that administration of a pharmaceutical composition comprising a one or more of the phosphatidyl-THIO conjugates could be used in conjunction with a treatment B, such as gene therapy, chemotherapeutic, radiotherapeutic, or immunotherapeutic intervention, in addition to other pro-apoptotic or cell cycle regulating agents. It also is contemplated that various standard therapies, as well as surgical intervention, may be applied in combination with the described telomere shortening and telomere dysfunction-inducing therapy.

a. Chemotherapy

Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

b. Radiotherapy

Radiotherapies can cause DNA damage and include what are commonly known as X-rays, and/or the directed delivery of radioisotopes to tumor cells. Other forms of DNA damaging factors are also contemplated such as microwaves and UV-irradiation.

c. Immunotherapy

Immunotherapeutics, generally, rely on the use of immune effector cells and molecules to target and destroy cancer cells. Immunotherapy, thus, could be used as part of a combined therapy, in conjunction with the administration of a pharmaceutical composition comprising a one or more of the phosphatidyl-THIO conjugates. Immunotherapy modality relates to the targeting of the tumor cell through some marker of the tumor cell that is amenable to targeting, i.e., is not present on the majority of other cells. Many tumor markers exist and any of these may be suitable for targeting with a second treatment modality in the context of the present disclosure. Common tumor markers include carcinoembryonic antigen, prostate specific antigen, urinary tumor associated antigen, fetal antigen, tyrosinase (p9′7), gp68, TAG-72, HMFG, Sialyl Lewis Antigen, MucA, MucB, PLAP, estrogen receptor, laminin receptor, erb B and p155

d. Surgery

Curative surgery is a cancer treatment that can be used in conjunction with a pharmaceutical composition comprising a one or more of the phosphatidyl-THIO conjugates. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). It is further contemplated that composition of the present disclosure can be administered in conjunction with removal of superficial cancers, precancers, or incidental amounts of normal tissue. Upon excision of part of all of cancerous cells, tissue, or tumor, a cavity may be formed in the body. Treatment may be accomplished by administration of a pharmaceutical composition comprising a one or more of the phosphatidyl-THIO conjugates.

e. Other Anti-Cancer Agents

It is contemplated that other anti-cancer agents may be used in combination with comprising a one or more of the phosphatidyl-THIO conjugates disclosed herein to additively or synergistically enhance the therapeutic efficacy of treatment.

These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other anti-cancer agents that increase the sensitivity of a hyperproliferative cell to apoptosis, and signal transduction inhibitors like the antibody c225, could be used in combination one or more of the phosphatidyl-THIO conjugates disclosed herein to improve the treatment efficacy.

Lastly, additional agents can also include anti-cancer agents which are broadly characterized as anti-metabolites, inhibitors of topoisomerase I and II, alkylating agents and microtubule inhibitors (e.g., taxol). Anti-cancer agents for use in the present invention include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Hormonal therapy may also be used in combination with the administration of a pharmaceutical composition may be used in combination with one or more of the phosphatidyl-THIO conjugates. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

The present disclosure provides for sequential treatment of cancers using the phosphatidyl-THIO conjugates followed by PD-L1, PD-1 and/or CTLA-4 therapy. The periods for each treatment may vary and it is contemplated that short gap between treatments will be advantageous. For example, for the phosphatidyl-THIO conjugates the treatment may be as little as 2 days but may be 3, 4 or more days, including 2-4 days. The gap prior to PD-L1, PD-1 and/or CTLA-4 treatment should be at least one day and may be up 14 days, such as 2-4 days. An overlap between the phosphatidyl-THIO conjugates and PD-L1, PD-1 and/or CTLA-4 might need to be avoided due to potentially detrimental effects of the phosphatidyl-THIO conjugates on activated effector T cells.

The daily dosage of phosphatidyl-THIO conjugates disclosed herein will be between 0.5 mg/kg and 10 mg/kg, preferably intravenous or oral. The dose of PD-L1, PD-1 and/or CTLA-4 will be consistent with approved current dosing schedules.

In the context of the present disclosure, it also is contemplated that the phosphatidyl-THIO conjugates and anti PD-L1 such as atezolizumab or a phosphatidyl-THIO conjugate and anti PD-1 such as Libtayo® or anti CTAL-4 could be used in conjunction with chemo- or radiotherapeutic intervention, or other treatments. It also may prove effective, in particular, to combine a phosphatidyl-THIO conjugate disclosed herein, anti PD-L1, anti PD-1 or anti CTLA-4 with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with a phosphatidyl-THIO conjugate disclosed herein and at least one other agent. These compositions would be provided in a sequential or combined amount effective to kill or inhibit proliferation of the cell. This process may involve contacting the cells with phosphatidyl-THIO conjugate/anti PD-L1, anti PD-1 or anti CTLA-4 and the other agent(s) or factor(s) at the same time. This may be achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the phosphatidyl-THIO conjugates/anti PD-L1, anti PD-1 or anti CTLA-4 therapy may precede or follow the other agent treatment by intervals ranging from minutes to weeks. In embodiments where the other agent and the interferon prodrugs are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where a phosphatidyl-THIO conjugates disclosed herein/anti PD-L1, anti PD-1 or anti CTLA-4 therapy is “A” and the other therapy is “B”, as exemplified below:

A/B/A B/A/B B/B/A A/A/B B/A/A A/B/B B/B/B/A B/B/A/B

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are contemplated for use with the present disclosure. Other chemotherapeutics include selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene. The agents camptothecin, actinomycin D, and mitomycin C are commonly used chemotherapeutic drugs. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m2 for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m2 at 21-day intervals for doxorubicin, to 35-50 mg/m2 for etoposide intravenously or double the intravenous dose orally. Microtubule inhibitors, such as taxanes, also are contemplated. These molecules are diterpenes produced by the plants of the genus Taxus and include paclitaxel and docetaxel.

Epidermal growth factor receptor inhibitors, such as Iressa, mTOR, the mammalian target of rapamycin (also known as FK506-binding protein 12-rapamycin associated protein 1 (FRAP1)), is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure. Another EGFR inhibitor of particular utility here is Gefitinib.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5 fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage to DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that a distinct immunotherapy, a hormone therapy, a toxin therapy and/or surgery can be used.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Preparation of 5′-phosphatidyl derivatives of 6TdG and 6-thioguanosine (6TG) Using Microbial Phospholipase I (PLD)

### Materials And Methods

Syntheses of phosphatidyl nucleotides was conducted in stirring biphasic reaction mixtures (10-35 ml, 37° C.) consisting of dichloromethane and 0.2 M sodium acetate buffer containing 0.15 M CaCl₂ (pH 6.0), in the ratio of 6/4, v/v. The reaction mixture also contained (per 1 ml): 5-10 μmol of nucleoside, 6-12 μmol of respective phosphatidylcholine (the molar ratio of nucleoside to phosphatidylcholine 1:1.2 to 1:1.6), and approx. 0.2 mg of PLD.

Products were isolated using Silica gel LiChroprep 60, 40-63 μm column.

The dry PLD enzyme was obtained from the culture of the Streptomyces netropsis BIM B-428D strain.

### Results

Sixteen liponucleotides containing phosphatidyl groups with saturated and unsaturated fatty acid residues of various lengths (C4-C18) were obtained. The compounds purity were at least 90% as determined by thin layer chromatography (TLC). Conversion rate of the nucleosides into liponucleotides ranged from 33 to 83 mol. % depending on the nucleoside and the phospholipid fatty acid compositions. The structure of the obtained compounds was confirmed by UV-spectroscopy and LC mass-spectrometry.

What is claimed is:

1. A THIO-Lipid conjugated prodrug comprising a compound of Formula I, 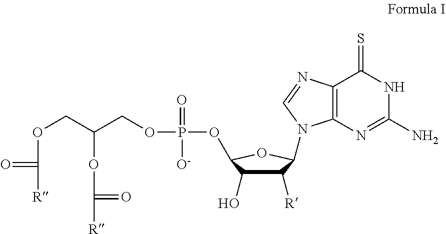
wherein R′ is H or OH and R″ is C₃-C₁₇.

2. A compound of claim 1, wherein the compound is selected from one of more of the following compounds:
5′-(1,2-dimyristoyl-sn-glycero-3-phospho)-6-thio-2′-deoxyguanosine,
5′-(1,2-dilauroyl-sn-glycero-3-phospho)-6-thio-2′-deoxyguanosine,
5′-(1,2-dilauroyl-sn-glycero-3-phospho)-6-thio-guanosine,
5′-(1,2-dioctanoyl-sn-glycero-3-phospho)-6-thio-2′-deoxyguanosine,
5′-(1,2-dioctanoyl-sn-glycero-3-phospho)-6-thio-guanosine,
5′-(1,2-dihexanoyl-sn-glycero-3-phospho)-6-thio-2′-deoxyguanosine,
5′-(1,2-dibutyryl-sn-glycero-3-phospho)-6-thio-2′-deoxyguanosine,
5′-(1,2-dihexanoyl-sn-glycero-3-phospho)-6-thio-guanosine,
5′-(1,2-dipalmitoyl-sn-glycero-3-phospho)-6-thio-2′-deoxyguanosine,
5′-(1,2-distearoyl-sn-glycero-3-phospho)-6-thio-2′-deoxyguanosine, and
5′-(1,2-dioleoyl-sn-glycero-3-phospho)-6-thio-2′-deoxyguanosine.

3. A compound of claim 1 or 2, wherein the compound has the following structure:

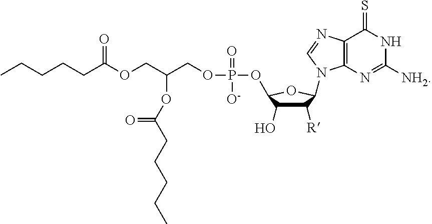

4. A pharmaceutical composition comprising a compound of any one of claims 1-3.

5. A pharmaceutical formulation comprising any one of claims 1-4, and a least one pharmaceutically acceptable excipient.

6. A process for preparing the pharmaceutical formulation of claim 5, comprising combining a compound of any one of claims 1-3 with at least one pharmaceutically acceptable excipient.

7. A method of treating a subject who has cancer, comprising administrating to the subject a compound of any one of claims 1-5, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.
